(19) United States
(12) Patent Application Publication
(10) Pub. No.: US 2021/0259276 A1
(43) Pub. Date: Aug. 26, 2021

(54) L-THEANINE FORMULATION

(71) Applicant: DSM IP ASSETS B.V., HEERLEN (NL)

(72) Inventors: Jose BARBOSA, Kaiseraugst (CH); Zhenbo MA, Kaiseraugst (CH); Conroy Clive SALMON, Kaiseraugst (CH); Sherry SITEMAN, Kaiseraugst (CH)

(21) Appl. No.: 17/179,071

(22) Filed: Feb. 18, 2021

Related U.S. Application Data
(60) Provisional application No. 62/978,979, filed on Feb. 20, 2020.

(51) Int. Cl. A23G 3/44; A23G 3/42; A23G 3/52; A23G 3/54; A23L 33/175; A23L 33/125

(52) U.S. Cl. CPC A23G 3/44; A23G 3/42; A23G 3/52; A61K 31/198; A23L 33/175; A23L 33/125; A23G 3/54

(57) ABSTRACT

The present invention relates to a new formulation comprising L-theanine.

The present invention relates to a new formulation comprising L-theanine.

L-theanine is an amino acid. The human body does not produce this compound, and it is not essential for humans, but it has some very positive effects on the human body as it is described below.

L-theanine can be found naturally in green tea, black tea, and certain types of mushroom.

L-theanine has very interesting positive effects when consumed.

L-theanine may affect the levels of certain chemicals in the brain. These include serotonin and dopamine, which influence mood, sleep, and emotion, and cortisol, which helps the body deal with stress. A change in the balance of these chemicals can change a person's mood or stress levels.

Furthermore, several studies have suggested that L-theanine could help people relax before bedtime, get to sleep more easily, and sleep more deeply.

These benefits may result from the specific effects that the amino acid has on brain chemicals that play a role in sleep.

Furthermore, research suggests that the L-theanine in green or black tea may contribute to this feeling of relaxation by reducing a person's resting heart rate.

Furthermore, there are studies that indicate the consumption of L-theanine could improve a person's mental and physical health. It seems to have neuroprotective effects, which improves brain function.

On its own, L-theanine may improve a person's attention and reaction times. In combination with caffeine, it may lead to improvements in their number skills and alertness.

Furthermore, L-theanine can create a savory taste, also known as umami flavor. Research suggests that umami flavors may reduce appetite, which often helps with weight loss.

Furthermore, L-theanine may support the body in fighting off illness. Several studies have suggested that taking L-theanine could boost a person's immune system, making them less likely to get common colds or the flu.

Furthermore, L-theanine may help reduce stress, and a reduction in stress and increased relaxation can lower the heart rate. In turn, this may help lower blood pressure.

Finally it was found that L-theanine may increase the effectiveness of a chemotherapy drug called doxorubicin. It is possible that it makes doxorubicin more effective by increasing the amount of the drug in cancer cells.

Due to the important and very positive of the effects L-theanine has there is always a need to provide formulation comprising L-theanine.

L-theanine has poor compressibility and flowability which causes problems in some formulations.

To deliver L-theanine in tablet format often faces challenges such as capping and low tablet hardness. Many commercially available tablets comprising L-theanine have a low L-theanine content (such as 100 mg per unit or even lower) There are also L-theanine capsules commercially available but the 2-piece capsule shell is often relatively large and not very easy for a consumer to swallow.

The goal of the present invention, was to find a formulation which allows to deliver L-theanine in high dosages (more than 100 mg per unit) and that the formulation is still easy to swallow.

Surprisingly it was found out that it is possible to create a formulation with a high amount of L-theanine in the form of a non-frozen aerated confectionery.

The present invention relates to a non-frozen confectionery (C), consisting of
(a) an aerated core and
(b) an optional coating covering the core,
   wherein the core comprises L-theanine.

In the context of the present invention we defined the core as the part of the formulation which can be coated, but the formulation can be a core as such (no coating).

The formulation comprises at least 70 mg L-theanine.

Preferably the L-theanine content is 70 mg to 250 mg.

Therefore, the present invention related to a non-frozen confectionery (C1), which is the non-frozen confectionery (C), wherein the L-theanine of the non-frozen confectionery is at least 70 mg.

Therefore, the present invention related to a non-frozen confectionery (C2), which is the non-frozen confectionery (C) or (C1), wherein the L-theanine of the non-frozen confectionery is 70 mg to 250 mg.

Aerated confectioneries can be classified into two sub-groups, grained and non-grained candies. Grained candies are made from supersaturated sucrose solutions while non-grained candies are made from unsaturated sucrose solutions. The basic composition for marshmallows of all kinds includes water, a disaccharide sugar such as sucrose, a monosaccharide sugar such as dextrose or corn syrup, and a whipping agent such as gelatin. Suitable flavor and coloring materials may also be incorporated into the basic composition. Among the many formulas that are known for marshmallow production are those found in the following: “Candy Making as a Science and as an Art,” by Claude D. Barnett, Don Gussow Publications, Inc., New York, 1960, pp. 99-103; “An Encyclopedia of Candy and Ice Cream Making,” by Simon I. Leon, Chemical Publishing Company, New York, 1969, pp. 294-304, and, “A Textbook on Candy Making,” by Alfred E. Leighton, The Manufacturing Confectioner Publishing Company, Oak Park, III., 1952, pp. 55-58. See-also, for example, U.S. Pat. No. 3,607,309, issued Sep. 21, 1961 to E. S. Olney et al, and U.S. Pat. No. 2,847,311, issued Aug. 12, 1958 to Alexander J. Doumak. Soft marshmallow or “long grain” marshmallow has an elastic and chewy consistency and is the type usually associated with the term “marshmallow”. Sugar ratios are so regulated so that crystallization of the sugars or “graining” does not take place within a reasonable period of time. To accomplish this reduction in the tendency to crystallize, corn syrup of various dextrose equivalents (“DE”) have been employed to inhibit the onset of crystallization.

The non-frozen confectionery according to the present invention consists of a core and an optional coating.

The core of the non-frozen coated confectionery according to the present invention is aerated. A preferred core is kind of a marshmallow (or when there is no coating then the non-frozen confectionery is a marshmallow).

Therefore, the present invention related to a non-frozen confectionery (C3), which is the non-frozen confectionery (C), (C1) or (C2), characterized in that the non-frozen confectionery is a marshmallow.

The aerated core, which comprises the L-theanine, usually (and preferably) comprises the following ingredients:
(i) sugar and/or sugar substitute,
(ii) water,
(iii) air,
(iv) at least one whipping agent/aerator (usually a protein)
(v) any flavor ingredient and/or other auxiliary agent.

Therefore, the present invention related to a non-frozen confectionery (C4), which is the non-frozen confectionery (C), (C1), (C2) or (C3), wherein the aerated core comprises the following ingredients:
(i) sugar and/or sugar substitute,
(ii) water,
(iii) air,
(iv) at least one whipping agent/aerator (usually a protein)
(v) any flavor ingredient and/or other auxiliary agent.

The sugar used in the aerated core of the confectionery according to the present invention can be any usually used sugar such as sucrose, corn syrup (glucose syrup) and/or invert sugar.

The aerated core comprises usually between 50-99 wt-%, based on the total weight of the core, of at least one sugar. Preferably the at least one sugar is chosen from the group consisting of sucrose, corn syrup (glucose syrup), tapioca syrup, and invert sugar.

Therefore, the present invention related to a non-frozen confectionery (C5), which is the non-frozen confectionery (C4), wherein the aerated core comprises between 50-99 wt %, based on the total weight of the core, of at least one sugar.

Therefore, the present invention related to a non-frozen confectionery (C5′), which is the non-frozen confectionery (C4), wherein the sugar used in the aerated core is chosen from the group consisting of sucrose, corn syrup (glucose syrup), tapioca syrup, and invert sugar.

The aerated core comprises water. The amount (in weight %, based on the total weight of the core) of water in the core is usually low. During the process of production of the aerated core, most of the water, which is used is evaporated. The aerated usually comprises up to 20 wt-%, based on the total weight of the core, of water. Usually it is less than 15 wt-% (preferably 1-10 wt-% of water).

Therefore, the present invention related to a non-frozen confectionery (C6), which is the non-frozen confectionery (C4), (C5) or (C5′), wherein the aerated core comprises up to 20 wt-%, based on the total weight of the core, of water.

Therefore, the present invention related to a non-frozen confectionery (C6′), which is the non-frozen confectionery (C4), (C5) or (C5′), wherein the aerated core comprises less than 15 wt-%, based on the total weight of the core, of water.

Therefore, the present invention related to a non-frozen confectionery (C6″), which is the non-frozen confectionery (C4), (C5) or (C5′), wherein the aerated core comprises 1-10 wt-%, based on the total weight of the core, of water.

The marshmallow is a foam, consisting of an aqueous continuous phase and a gaseous dispersed phase (in other words, a liquid with gas bubbles spread throughout). In addition to be a foam, this also makes marshmallows an “aerated” confectionery because it is made up to of 50% (by volume) of air. It is also possible to use other gases than air. But air is the most common one.

Therefore, the present invention related to a non-frozen confectionery (C7), which is the non-frozen confectionery (C4), (C5), (C5′), (C6), (C6′) or (C6″), wherein the aerated core comprises up to 50% (by volume) of air.

The whipping agent/aerator (iv) used to produce the marshmallow core is usually and preferably a protein (or a mixture of proteins). Preferred proteins are albumen (egg whites) and gelatin.

Albumen is a mixture of proteins found in egg whites and is utilized for its capacity to create foams.

Gelatin is made up of collagen, a structural protein derived from animal skin, connective tissue, and bones.

The aerated core usually comprises between 1-5 wt-%, based on the total weight of the core, of at least one whipping agent/aerator. Preferably the whipping agent/aerator is a protein. More preferably are albumen (egg whites) and/or gelatin.

Therefore, the present invention related to a non-frozen confectionery (C8), which is the non-frozen confectionery (C4), (C5), (C5′), (C6), (C6′), (C6″) or (C7), wherein the aerated core comprises between 1-5 wt-%, based on the total weight of the core, of at least one whipping agent/aerator.

Therefore, the present invention related to a non-frozen confectionery (C8′), which is the non-frozen confectionery (C8), (C5), (C5′), (C6), (C6′), (C6″), (C7) or (C8), wherein the whipping agent/aerator is a protein. More preferably are albumen (egg whites) and/or gelatin.

Therefore, the present invention related to a non-frozen confectionery (C8″), which is the non-frozen confectionery (C8), (C5), (C5′), (C6), (C6′), (C6″), (C7) or (C8), wherein the whipping agent/aerator is chosen from the group consisting of albumen (egg whites) and gelatin.

The essential feature of the aerated core of the non-frozen confectionery according to the present invention is the presence of L-theanine.

The amount of L-theanine can vary depending on the size and on the field of application of the formulation. Preferably L-theanine is used in amount of around 200 mg. This means that the formulation comprises 200 mg of L-theanine. It can also comprise more or less.

The aerated core of the non-frozen confectionery according to the present invention can comprise additional ingredients, such as flavors, cocoa, acids, colors, starches, etc. The amount of these (non-essential for the invention) ingredients can vary.

The non-frozen confectionery according to the present invention optionally has a coating. This coating (layer) an cover the aerated core in total.

The coating is usually (and preferably) a powder coating. Such a powder coating has no essential effect on the core. It can be used for visual (appearance of the formulation) and/or taste reasons.

A very suitable and also preferred (powder) coating is a coating made from chocolate (or cacao) powder.

These coatings are well-known in the prior art.

Any known process (as used in the food or pharma industry can be used).

The thickness of the coating is usually up to 2 mm. Preferably (and ideally) the coating covers the aerated core equally.

The ratio (regarding the weight) of the core and the coating of the non-frozen coated confectionery according to the present invention is from 1:0.0001 to 1:2. This ratio depends strongly on the fluffiness of the core. The more air is in the core the more the ratio shifts to the coating.

The overall size as well as the shape of one non-frozen coated confectionery can vary. A usual and common form is sphere-like. The size (the longest diameter) if such a sphere is usually from 0.5 cm-3 cm.

But the shape can also be like a drum or any other form, which is appealing to be consumed.

The process of production of the non-frozen coated confectionery with an aerated core is usually the following
(1) the aerated core is produced and then
(2) the aerated core is coated (by a powder coating).

Process of Production of the Aerated Core

The aerated cores can be produced according to well-known process. A suitable way is the following:
(I) Gelatin is added to water and heated in with water bath (˜60° C.). Allow at least 40 mins for hydration.
(II) In another pot, heat corn syrup with water and granulated sugar to 108° C.
(III) Pour cooked syrup into Hobart mixing bowl, then set mixer speed at medium using the whisk for mixing.
(IV) Add hydrated gelatin from step (I) and mix it. Slowly add the following: Egg Albumin, Malic acid, Tapioca starch, powdered sugar. Mix for a few minutes then add the L-theanine and/or other dietary ingredients. Temperature is about 50° C.
(V) Finally add flavor (if needed) and mix for another few mins. Form core marshmallows with target serving size after cooling down to room temperature.

Coating (Powder Coating) of the Aerated Core

The coating of the aerated cores (which are produced as disclosed above) can be produced according to well-known process.

The following examples serve to illustrate the invention.

### EXAMPLES

### Example 1

Mix gelatin in water (part 2) with water bath (˜60° C.). Allow at least 40 min for hydration
Cook syrup with water (part 1), granulated sugar, and corn syrup in a pot to 108° C.
Pour syrup into Hobart mixing bowl and set mixer (whisk) speed to medium
Add hydrated gelatin from step 1 and mix for a min. Slowly add egg albumin, malic acid, tapioca starch, powdered sugar, cocoa powder, honey powder, steviol glycosides. Mix for 5 min (high speed) then add premix which comprises L-theanine, caffeine, vitamin B6, and vitamin B12.

Temperature of the mixture is about 50° C.
Add flavor and mix for another 5 min. Form core marshmallows with target serving size and dust with Sweet Ground Chocolate and Cocoa Gourmet Powder (estimate 20 mg powder/serving)

The following table is a typical composition (produced according to Example 1):

| Ingredient | Amount (mg) |
|---|---|
| L-Theanine | 224.49 |
| Pyridoxine Hydrochloride | 1.03 |
| Vitamin B12 0.1% WS | 1.56 |
| Caffeine 60% encapsulated | 97.17 |
| Sugar Icing | 75.75 |
| Malic Acid | 13.29 |
| Egg Albumin | 36.25 |
| Water (part 1) | 181.27 |
| Sugar | 1191.55 |
| Corn Syrup | 589.72 |
| Water (part 2) | 120.67 |
| Gelatin | 54.85 |
| Tapioca Starch | 16.92 |
| Honey Powder | 28.00 |
| Steviol glycosides | 2.00 |
| Powdered Sugar | 1179.66 |
| Nat. Cocoa Powder | 290.03 |
| Nat Chocolate Flavor | 7.25 |
| Solid weight (core) | 3809.52 |
| Sweet Ground Chocolate and Cocoa | 20.00 |
| Gourmet Powder (powder coating) | |

1. A non-frozen confectionery consisting of
(a) an aerated core and
(b) an optional coating covering the core,
wherein the core comprises L-theanine.

2. The non-frozen confectionery according to claim 1 comprising at least 70 mg L-theanine.

3. The non-frozen confectionery according to claim 1 comprising 70 mg to 250 mg L-theanine.

4. The non-frozen confectionery according to claim 1, characterized in that the non-frozen confectionery is a marshmallow.

5. The non-frozen confectionery according to claim 1, wherein the aerated core comprises
(i) sugar and/or sugar substitute,
(ii) water,
(iii) air,
(iv) at least one whipping agent/aerator (usually a protein)
(v) any flavor ingredient and/or other auxiliary agent.

6. The non-frozen confectionery according to claim 5, wherein the aerated core comprises between 50-99 wt-%, based on the total weight of the core, of at least one sugar.

7. The non-frozen confectionery according to claim 5, wherein the sugar is chosen from the group consisting of sucrose, corn syrup (glucose syrup), tapioca syrup, and invert sugar.

8. The non-frozen confectionery according to claim 5, wherein the aerated core comprises up to 20 wt-%, based on the total weight of the core, of water.

9. The non-frozen confectionery according to claim 5, wherein the aerated core comprises less than 15 wt-%, based on the total weight of the core, of water.

10. The non-frozen confectionery according to claim 5, wherein the aerated core comprises 1-10 wt-%, based on the total weight of the core, of water.

11. The non-frozen confectionery according to claim 5, wherein the aerated core comprises up to 50% (by volume) of air.

12. The non-frozen confectionery according to claim 5, wherein the aerated core comprises between 1-5 wt-%, based on the total weight of the core, of at least one whipping agent/aerator.

13. The non-frozen confectionery according to claim 5, wherein the whipping agent/aerator is a protein.

14. The non-frozen confectionery according to claim 5, wherein the whipping agent/aerator is chosen from the group consisting of albumen (egg whites) and gelatin.
